(19) United States
(12) Patent Application Publication
(10) Pub. No.: US 2024/0301454 A1
(43) Pub. Date: Sep. 12, 2024

(54) Method for Producing Phycocyanobilin Using a Recombinant Escherichia Coli

(71) Applicant: Jiangnan University, Wuxi (CN)

(72) Inventors: Jingwen Zhou, Wuxi (CN); Jian Chen, Wuxi (CN); Yuqi Wang, Wuxi (CN); Sha Xu, Wuxi (CN); Weizhu Zeng, Wuxi (CN)

(21) Appl. No.: 18/428,893

(22) Filed: Jan. 31, 2024

(30) Foreign Application Priority Data
Feb. 10, 2023 (CN) 2023100955399

(51) Int. Cl. C12P 17/16; C12N 9/02; C12N 15/70

(52) U.S. Cl. CPC C12P 17/165; C12N 9/001; C12N 9/0071; C12N 15/70; C12Y 103/07005; C12Y 114/14; C12N 2800/101

(57) ABSTRACT

Disclosed is a method for producing phycocyanobilin by use of a recombinant Escherichia coli that express heterologous heme oxygenase ho1 and ferredoxin oxidoreductase pcyA derived from Synechocystis sp. PCC6803. According to the present disclosure, heterologous expression of ho1 and pcyA genes leads to conversion of heme to an intermediate biliverdin for phycocyanobilin synthesis, and reduces the accumulation of biliverdin in the process of the phycocyanobilin synthesis. The genome of E. coli is further engineered to overexpress related genes of a metabolic pathway of phycocyanobilin, and a strain of recombinant E. coli with high yield of phycocyanobilin is obtained. The recombinant E. coli strain is cultured for 36 hr in a system using glycerol as a substrate, and the phycocyanobilin yield can reach 147 mg/L.

### CROSS-REFERENCE TO RELATED APPLICATIONS

This application claims the benefit of Chinese patent application No. 2023100955399, filed Feb. 10, 2023, the content of which is incorporated by reference herein.

### BACKGROUND OF THE INVENTION

### Field of the Invention

The present invention relates to a method for producing phycocyanobilin by use of a recombinant Escherichia coli, belonging to the technical fields of genetic engineering and bioengineering.

### Description of the Related Art

Phycocyanobilin is a natural pigment of some algae. Due to its special spectroscopic properties, it is widely used as an excellent fluorescent probe in the fields such as immune disease diagnosis and photodynamic therapy. Furthermore, because of its anti-inflammatory activity, antioxidant activity, ability to eliminate free radicals and other functions, it also has great potential for applications in the field of food, cosmetics, and medical and healthcare products. As an important industrial microorganism, E. coli has many advantages such as fast reproduction speed, clear genetic background, and mature gene editing technology, and is an important host strain for heterologous protein expression. Therefore, E. coli is an ideal host strain to be used for synthesis of the phycocyanobilin. Biosynthesis of the phycocyanobilin starts from 5-aminolevulinic acid (ALA). A synthesis pathway of the ALA in the E. coli is a pathway C5, and glutamic acid is transformed into the ALA through the pathway C5. Two molecules of ALA are catalyzed by porphobilinogen synthase to generate one molecule of porphobilinogen (PBG), four molecules of PBG are catalyzed by porphobilinogen deaminase to form 1-hydroxymethylbilane (HMB), and the 1-hydroxymethylbilane (HMB) is cyclized by uroporphyrinogen synthase to form uroporphyrinogen III; subsequently, the decarboxylation and oxidation reactions catalyzed by porphyrinogen III decarboxylase, co-porphyrinogen III oxidase, and protoporphyrinogen oxidase convert porphyrinogen III to protoporphyrin IX; and finally, ferrochelatase intercalates iron ions into protoporphyrin to form heme. After the formation of heme, heme oxygenase (Ho1) degrades heme to biliverdin IX by a redox reaction using oxygen, which releases one molecule of CO. After the biliverdin is catalyzed by ferredoxin oxidoreductase (PcyA), free phycocyanobilin (Free PCB) is finally generated.

At present, the phycocyanobilin is mainly extracted from spirulina. Crude phycocyanobilin is obtained by carrying out pyrolysis on the spirulina with methanol, and then purer phycocyanobilin is obtained through separation and purification. However, the long period of pyrolysis by the methanol not only requires a large amount of spirulina as a raw material, but also consumes a large amount of energy to provide heat. Therefore, there is a need for a genetic engineering technology in which the single and purer PCB can be extracted by fermentation of recombinant E. coli, thereby avoiding the complicated process of extracting the phycocyanobilin from the spirulina, and reducing the cost of spirulina pyrolysis.

### SUMMARY OF THE INVENTION

The present invention provides a recombinant strain conducive to accumulation of phycocyanobilin, which reduces production costs of the phycocyanobilin and has the potential for industrial production.

In one embodiment of the invention, it provides a strain of recombinant E. coli for synthesis of phycocyanobilin, which heterologously expresses ho1 and pcyA genes derived from Synechocystis sp. PCC6803 using BL21 (DE3) as an original strain.

In one embodiment, the ho1 and pcyA genes are expressed by using pRSFDuet-1 as an expression vector.

In one embodiment, the ho1 and pcyA genes are expressed through fusion of short peptide tags RIDD and RIAD.

In one embodiment, the recombinant E. coli further enhances synthesis of heme.

In one embodiment, the recombinant E. coli further undergoes at least one of the following improvements:

(1) overexpression of endogenous genes hemB (Gene ID: 945017, SEQ ID NO:7), hemC (Gene ID: 947759, SEQ ID NO:8), and hemD (Gene ID: 948587, SEQ ID NO:9) in the E. coli BL21 (DE3); and

(2) overexpression of endogenous genes hemE (Gene ID: 948497, SEQ ID NO:10), hemF (Gene ID: 946908, SEQ ID NO:11), hemG (Gene ID: 948331, SEQ ID NO:12), and hemH (Gene ID: 947532, SEQ ID NO:13) in the E. coli BL21 (DE3).

In one embodiment, according to the recombinant E. coli, under conditions that the BL21 (DE3) is used as the original strain and the pRSFDuet-1 is used as the expression vector, a gene encoding the heme oxygenase ho1 as shown in SEQ ID NO:1 and a gene encoding the ferredoxin oxidoreductase pcyA as shown in SEQ ID NO:2 are expressed, and the endogenous genes hemB, hemC, and hemD as well as the endogenous genes hemE, hemF, hemG, and hemH in the E. coli BL21 (DE3) are overexpressed; and the heme oxygenase ho1 and the ferredoxin oxidoreductase pcyA are expressed through fusion of short peptide tags RIDD as shown in SEQ ID NO:3 and RIAD as shown in SEQ ID NO:4.

In one embodiment, integrative expression of the gene hemB derived from the E. coli results in asymmetric condensation of ALA into a mono-pyrrole compound porphobilinogen.

In one embodiment, integrative expression of the gene hemC derived from the E. coli results in conversion of the porphobilinogen into destabilized linear tetrapyrrole, that is, hydroxymethylcholane.

In one embodiment, integrative expression of the gene hemD derived from the E. coli results in cyclization of the hydroxymethylcholane into tetrapyrrole-ring uroporphyrinogen III.

In one embodiment, integrative expression of the gene hemE derived from the E. coli decarboxylates four acetate chains of the uroporphyrinogen III to form coproporphyrinogen III.

In one embodiment, integrative expression of the gene hemF derived from the E. coli decarboxylates the coproporphyrinogen III to produce protoporphyrinogen IX.

In one embodiment, integrative expression of the gene hemG derived from the E. coli allows the protoporphyrinogen IX to be oxidized to protoporphyrin IX by mediating six electrons.

In one embodiment, integrative expression of the gene hemH derived from the E. coli chelates ferrous ions into the macrocycle of the protoporphyrin IX to form heme.

In one embodiment, the short peptide tags RIDD and RIAD are fused at the C-terminal of the Ho1 and the N-terminal of the PcyA, so that a multi-enzyme complex is constructed, and the synthesis of phycocyanobilin is further enhanced.

In one embodiment, the gene hemEFGH is integrated at a position where a heme degrading gene yfeX is located.

In one embodiment, the gene hemBCD is integrated at an arsB site of an arsenate transporter gene.

In one embodiment, nucleotide sequences of genes encoding the heme oxygenase ho1 and the ferredoxin oxidoreductase pcyA are respectively shown in SEQ ID NO:1 and SEQ ID NO:2.

In one embodiment, nucleotide sequences of the short peptide tags RIDD and RIAD are as shown in SEQ ID NO:3 and SEQ ID NO:4, respectively.

In one embodiment, a nucleotide sequence of the gene hemBCD encoding the genes hemB, hemC and hemD is shown in SEQ ID NO:5.

In one embodiment, a nucleotide sequence of the gene hemEFGH encoding the genes hemE, hemF, hemG and hemH is shown in SEQ ID NO:6.

In one embodiment of the invention, it provides a method for producing phycocyanobilin, wherein the recombinant E. coli above is fermented to produce the phycocyanobilin.

In one embodiment, the recombinant E. coli is inoculated into a fermentation system and cultured at 35-37° C. for 2-3 hr, 0.5 mM IPTG is added, synthesis of the phycocyanobilin is induced and carried out at 25° C. and 200-220 rpm, and fermentation is carried out for 24-48 hr.

In one embodiment, it provides a fermentation medium containing 6 g KH₂PO₄, K₂HPO₄·3H₂P, (NH₄)₂SO₄, anhydrous citric acid, MgSO₄, yeast powder, glycerol, maltodextrin, vitamin B1, and a trace element solution.

In one embodiment, the trace solution contain Fe(III) citrate, ZnCl₂, MnSO₄·H₂O, CuSO₄·5H₂O, Na₂MoO₄·2H₂O, CaCl₂·2H₂O, H₃BO₃, CoCl₂·6H₂O, and NiSO₄·6H₂O.

### Beneficial Effects

According to the present disclosure, heterologous expression of ho1 and pcyA genes derived from Synechocystis sp. PCC6803 converts heme to an intermediate biliverdin for phycocyanobilin synthesis, and reduces accumulation of the intermediate product biliverdin for the phycocyanobilin synthesis. A genome of the E. coli is further transformed, and a strain of recombinant E. coli with high yield of phycocyanobilin is obtained by overexpressing the related genes in a metabolic pathway of the phycocyanobilin. The recombinant strain is cultured for 36 hr in a system with cheap glycerol as a substrate, and the phycocyanobilin yield can reach 147 mg/L.

### BRIEF DESCRIPTION OF THE DRAWINGS

FIG. 1 shows a plasmid map of a recombinant vector pRSFDuet-ho1-pcyA.

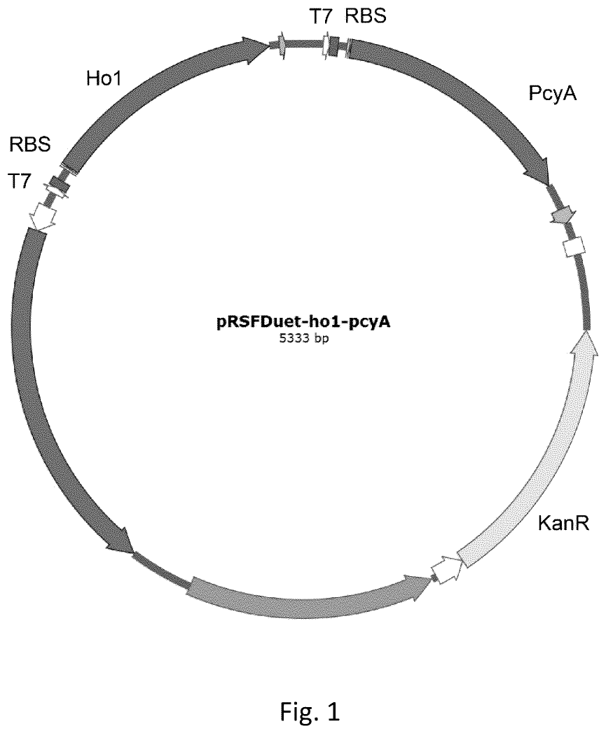

FIG. 2 shows a plasmid map of a recombinant vector pRSFDuet-ho1-RIDD-RIAD-pcyA.

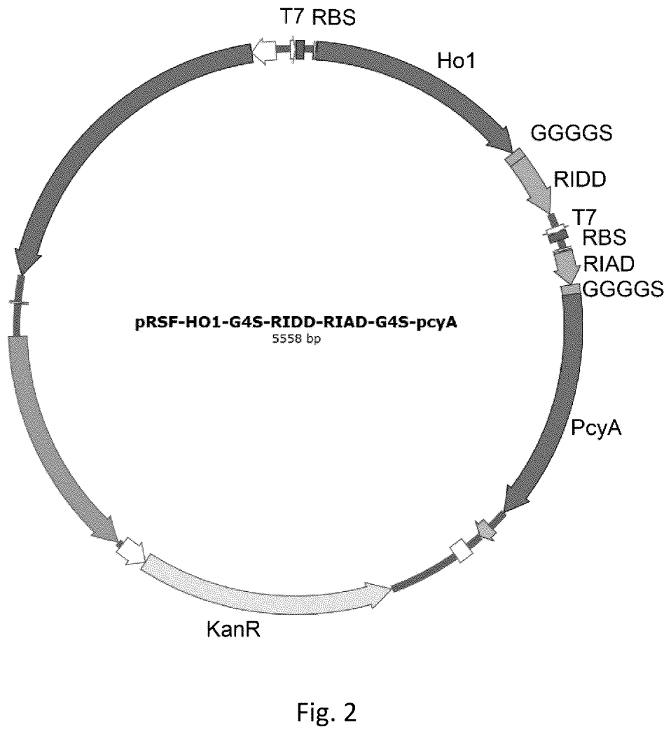

FIG. 3 shows a synthetic route diagram of phycocyanobilin synthesized by using E. coli.

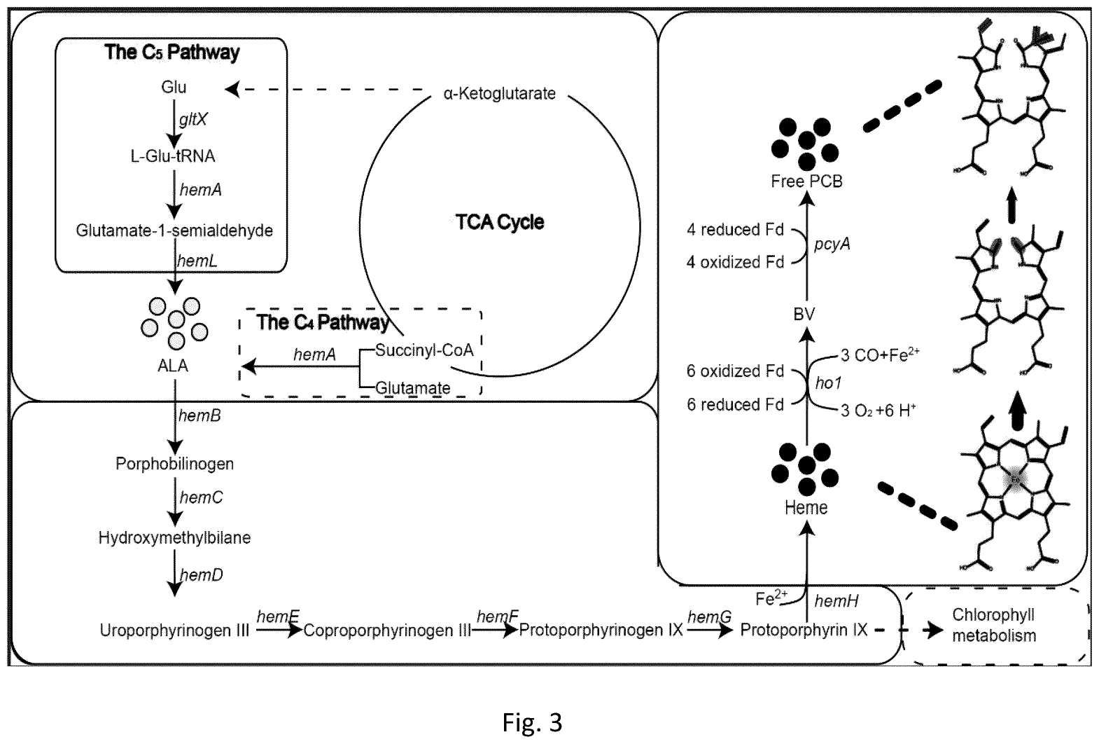

FIG. 4 shows an amount of phycocyanobilin synthesized by shake-flask fermentation of recombinant E. coli.

FIG. 5 shows LC-MS diagrams of recombinant E. coli, control, and standard.

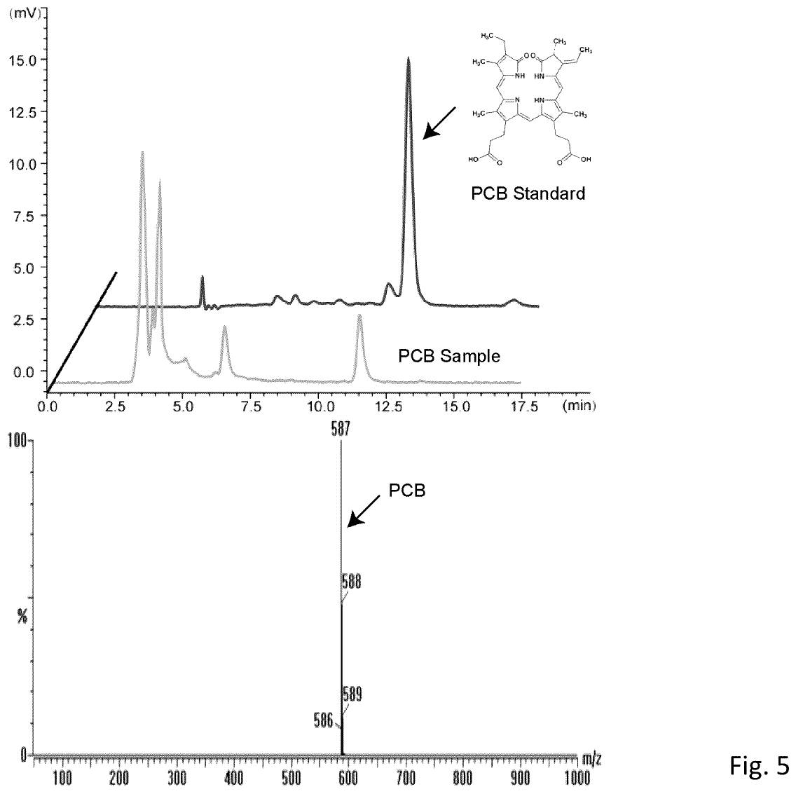

FIG. 6 shows an amount of phycocyanobilin synthesized in a 5 L fermentor.

### DETAILED DESCRIPTION OF THE INVENTION

### (1) Medium

LB medium: 5 g/L yeast extract powder, 10 g/L peptone, and 10 g/L sodium chloride. 15 g/L agar was added during the preparation of the LB solid medium.

Fermentation medium (per L): 6 g KH₂PO₄, 16.4 g K₂HPO₄·3H₂O, 5 g (NH₄)₂SO₄, 1 g anhydrous citric acid, 1 g MgSO₄·7H₂O, 10 g yeast powder, 30 g glycerol, 10 g maltodextrin, 0.1 g vitamin B1, and 1 mL a trace element solution.

Trace element solution: 100 g/L Fe(III) citrate, 18 g/L ZnCl₂, 14.64 g/L MnSO₄·H₂O, 0.75 g/L CuSO₄·5H₂O, 2 g/L Na₂MoO₄·2H₂O, 2 g/L CaCl₂·2H₂O, 3.0 g/L H₃BO₃, 2.5 g/L CoCl₂·6H₂O, 2.5 g/L NiSO₄·6H₂O, and 100 mL HCl.

(2) PCR reaction system and amplification conditions: a forward primer (10 μM): 1 μL, a reverse primer (10 μM): 1 μL, template DNA: 10-50 ng, 2×Phanta Max Master Mix: 25 μL, and double distilled water added to 50 μL. Amplification conditions: pre-deformation at 95° C. for 3 min, followed by 30 cycles (at 95° C. for 15 s, at 55° C. for 15 s, at 72° C. for 15 s), with an extension at 72° C. for 5 min.

A seamless cloning reaction system was as follows: 40 ng a target gene, 100 ng a vector added, 5 μL reaction enzyme mixture, and sterile ultrapure water added to make up to 10 μL. The reaction conditions were as follows: a reaction was performed at 50° C. for 60 min, and the product was placed on ice immediately after the reaction was finished. 10 μL of the product was transformed into E. coli competent cells JM109.

(3) The Competent Cell Preparation Kit of TaKaRa (TaKaRa, Dalian, China) was used to prepare the E. coli competent cells. Please refer to the instructions for the specific operation process. The prepared competent cells were preserved at −80° C., and subsequently could be transformed into plasmids, fragments, or the like.

(4) Chemical transformation method for E. coli: E. coli JM109 was streaked on a solid LB plate and cultured at 37° C. for 12 hr; single colonies were selected and inoculated into a liquid LB medium, allowed to grow at 37° C. and 220 rpm for 10 hr, and then transferred and inoculated into a fresh 25 mL liquid LB medium at an inoculation amount of 1%; the inoculated colonies were incubated at 37° C. for 1.5-2 hr; and when the OD₆₀₀ grew to 0.6-1, the cells were collected to produce competent cells.

(5) HPLC detection of phycocyanobilin: a chromatographic column (250×4.6 mm, m, Thermo-Fisher, Waltham, MA) was employed, and a concentration of the phycocyanobilin was determined at 380 nm using a high performance liquid chromatography system (Waters Corporation, Milfold, MA) and a UV detector (Waters 2487). An initial mobile phase consisted of 40% solvent A (acetonitrile containing 0.1% trifluoroacetic acid) and 60% solvent B (0.1% trifluoroacetic acid in water). The solvent gradient: 40% solvent B 0 min, 55% solvent B 30 min with a flow rate of 0.8 mL/min. Column temperature: 25° C.

(6) The strain information is shown in Table 1:

TABLE 1
Strains and genes used in the present disclosure

| Name of | |
|---|---|
| strain | Genotype |
| St07 | E. coli BL21 (DE3), pRSFDuet-T7lac-ho1^{S}-T7lac-pcyA^{S} |
| St17 | E. coli BL21 (DE3) ΔarsB::hemBCD |
| St18 | St 17, pRSFDuet-T7lac-ho1^{S}-T7lac-pcyA^{S} |
| St19 | E. coli BL21 (DE3) ΔarsB::hemBCD ΔyfeX::hemEFGH |
| St20 | St19, pRSFDuet-T7lac-ho1^{S}-T7lac-pcyA^{S} |
| St26 | St19, pRSFDuet-T7lac-ho1^{S}-GGGGS-RIDD-RIAD-GGGGS- |
| | T7lac-pcyA^{S} |

### Example 1. Synthesis of Key Genes for Phycocyanobilin Biosynthesis

Suzhou Jinweizhi Biotechnology Co., Ltd. carried out full-length chemical synthesis of the following genes: a heme oxygenase Ho1 gene (KEGG accession number sll1184) derived from Synechocystis sp. PCC 6803, with a nucleotide sequence as shown in SEQ ID NO:1, a phycocyanobilin gene derived from Synechocystis sp. PCC 6803, a ferredoxin oxidoreductase gene PcyA (KEGG accession number slr0116), with a nucleotide sequence as shown in SEQ ID NO:2, a nucleotide sequence as shown in SEQ ID NO:3 that encodes a short peptide tag RIDD, and a nucleotide sequence as shown in SEQ ID NO:4 that encodes a short peptide tag RIAD.

### Example 2. Construction of Gene Expression Cassettes Related to Biosynthesis Pathway of Phycocyanobilin

E. coli BL21(DE3) was used as an original strain for the synthesis of phycocyanobilin. First, in order to construct a synthesis pathway of the phycocyanobilin, a synthetic sequence of ho1 as shown in SEQ ID NO:1 was used as a template, and a primer pair F1/R1 was used for PCR amplification. Phanta MasterMix (Vazyme Company, Nanjin, China) high-fidelity pfu enzyme was selected for pre-denaturation at 95° C. for 3 min. The amplification phase consisted of 30 cycles, which were carried out at 95° C. for 15 s, 55° C. for 15 s, and 72° C. for 30 s, with an extension at 72° C. for 5 min. The PCR product was purified, and a fragment ho1 was obtained. A vector pRSFDuet-1 was used as a template, and a primer pair F2/R2 was used for PCR amplification. Phanta MasterMix high-fidelity pfu enzyme was selected for pre-denaturation at 95° C. for 3 min. The amplification phase consisted of 30 cycles, which were carried out at 95° C. for 15 s, 55° C. for 15 s, and 72° C. for 2 min, with an extension at 72° C. for 5 min. The PCR product was purified. The fragment ho1 and the vector pRSFDuet-1 were recombined into a vector pRSFDuet-ho1 by way of seamless cloning assembly, and transformed into E. coli JM109. The obtained vector was sent to Shanghai Sangon Biotech for sequencing, and the correct recombinant vector pRSFDuet-ho1 was obtained after the comparison was correct.

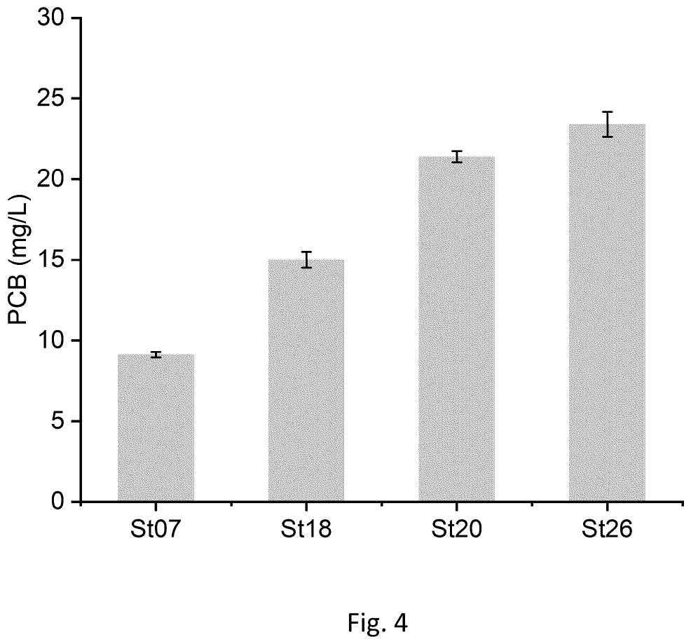

A synthetic sequence of pcyA as shown in SEQ ID NO:2 was used as a template, and a primer pair F3/R3 was used for PCR amplification. Phanta MasterMix high-fidelity pfu enzyme was selected for pre-denaturation at 95° C. for 3 min. The amplification phase consisted of 30 cycles, which were carried out at 95° C. for 15 s, 58° C. for 15 s, and 72° C. for 30 s, with an extension at 72° C. for 5 min. The PCR product was purified, and a fragment pcyA was obtained. The recombinant vector pRSFDuet-ho1 was used as a template, and a primer pair F4/R4 was used for PCR amplification. Phanta MasterMix high-fidelity pfu enzyme was selected for pre-denaturation at 95° C. for 3 min. The amplification phase consisted of 30 cycles, which were carried out at 95° C. for 15 s, 58° C. for 15 s, and 72° C. for 3 min, with an extension at 72° C. for 5 min. The PCR product was purified, and a vector fragment pRSFDuet-ho1 was obtained. The fragment pcyA and the vector fragment pRSFDuet-ho1 were recombined into a vector pRSFDuet-ho1-pcyA by way of seamless cloning assembly, and the recombined vector was transformed into E. coli JM109. The obtained vector was sent to Shanghai Sangon Biotech for sequencing, and the correct recombinant vector pRSFDuet-ho1-pcyA was obtained after the correct sequence was confirmed. The recombinant vector pRSFDuet-ho1-pcyA was transformed into E. coli BL21(DE3) to obtain an engineered strain St07. The engineered strain was cultured in a seed medium at 37° C. and 220 r/min for 12 hr to obtain a seed solution (with OD₆₀₀ being 3±0.2), and then the seed solution was inoculated into a fermentation medium containing kanamycin with a final concentration of 50 μg/mL at an inoculation concentration of 2%. After incubation at 37° C. and 220 rpm for 3 hr, IPTG with a final concentration of 0.5 mM was added. The synthesis of the phycocyanobilin was induced and carried out at 25° C. and 220 rpm, and the fermentation period was 48 hr. As shown in FIG. 4, the yield of the phycocyanobilin in the fermentation broth of the St07 was 9.1 mg/L.

TABLE 2
Primer sequence

| | Sequence 5′-3′ | |
|---|---|---|
| Primer | (Underlined for the homologous arm region) | SEQ ID NO. |
| F1 | TAAGGAGATATAATGAGCGTTAACCTGGCGAGCCA | SEQ ID NO: 14 |
| R1 | TTGGTCTGGCGACCTCTGAAGGCTAACGAACAGAAA | SEQ ID NO: 15 |
| | GTAATCGTATTGTA | |
| F2 | CGAACAGAAAGTAATCGTATTGT | SEQ ID NO: 16 |
| R2 | AGGTTAACGCTCATTATATCTCCTTATTAAAGTTAAA | SEQ ID NO: 17 |
| | CAAAATT | |
| F3 | ATATAATGGCGGTTACCGATCTGTCTCTGA | SEQ ID NO: 18 |
| R3 | GTTCTGTTCGATGTTATCCAGTAAGCAGATCTCAATT | SEQ ID NO: 19 |
| | GGATATCGGCCG | |
| F4 | ATTAGTTAAGTATAAGAAGGAGATATAATGGCGGTT | SEQ ID NO: 20 |
| | ACCGATCTGTC | |
| R4 | GCAGATCTCAATTGGATATCGGCCG | SEQ ID NO: 21 |

### Example 3. Enhancement of Endogenous Heme Synthesis Pathway

Since heme is a direct precursor for phycocyanobilin synthesis, an endogenous gene hemBCD (with a nucleotide sequence as shown in SEQ ID NO:5) of the E. coli was overexpressed and integrated at an arsB site of an arsenate transporter gene of E. coli so as to increase the supply of heme.

A primer pair F5/R5 was used to amplify a gene fragment PT7-lacO-RBS-hemB-PT7-lacO-RBS-hemCD with plasmid pRSF-hemBCD (published in the paper: Xinrui Zhao, Kyeong Rok Choi, Sang Yup Lee*. Metabolic engineering of Escherichia coli for secretory production of free haem. Nature Catalysis. 2018, 1: 720-728, with a nucleotide sequence as shown in SEQ ID NO:58). Using an E. coli BL21 (DE3) genome as a template, primer pairs F6/R6 and F7/R7 were used to amplify about 500 bp homologous arms U1 and D1 upstream and downstream of the arsB site, respectively, and the fragments were purified. Using the purified fragments U1 and D1 as templates, an integration cassette U-PT7-lacO-RBS-hemB-PT7-lacO-RBS-hemCD-D1 was stepwise obtained by amplification with primer pairs F8/R8 and F9/R9, and the fragment was purified. In order to obtain pTarget-arsB for the integration at the arsB site, pTarget was used as a template, a primer F10/R10 was used for amplification, and the fragment was purifie. The purified fragment was transformed into E. coli JM109, the plasmid was extracted and sequenced for verification, and a correct recombinant vector pTarget-arsB was obtained.

In order to prepare the E. coli BL21 (DE3) electrocompetent cells containing pCas, the plasmid pCas was transformed into E. coli BL21 (DE3) chemical competent cells. Single clones obtained from the transformation were picked and put into a 4 mL LB medium, kanamycin with a final concentration of 50 μg/mL was added, and incubation was carried out at 30° C. for 12 hr. Then, the bacterial liquid was inoculated into a 50 mL LB medium at an inoculation concentration of 2%, and the kanamycin with the final concentration of 50 μg/mL and a 10 mM arabinose solution were added. After incubation at 30° C. and 220 rpm for 4-6 hr, when OD₆₀₀ reached 0.6, the bacterial liquid was transferred to a 50 ml centrifuge tube, and stood still on ice for 15 min. Centrifugation was carried out at 4° C. and 4000 rpm for 10 min to remove supernatant, and 10 mL of 10% glycerol was added for resuspending; the operation was repeated twice, and the product was packaged according to 100 μL/part and then stored at −80° C. 400 ng recombinant vector pTarget-arsB and 1200 ng integration cassette U-PT7-lacO-RBS-hemB-PT7-lacO-RBS-hemCD-D1 were added into the E. coli BL21 (DE3) electrocompetent cells, and the mixture stood still on ice for 10 min, was then transferred into a 1 mm electric cup that had been pre-cooled for 10 min, and was subjected to electric shock with a voltage of 1.8 kv. After the electric shock, the product was put into a 1 ml LB liquid medium, and cultured at 30° C. for 1.5 hr. A primer pair F11/R11 was used for colony PCR verification. Verified single clones were used to knockout pTarget-arsB and pCas9 according to a published method, and an engineered strain E. coli BL21 (DE3) ΔarsB::hemBCD was thus obtained. The recombinant vector pRSFDuet-ho1-pcyA was transformed into E. coli BL21(DE3) ΔarsB::hemBCD to obtain the engineered strain St18. The engineered strain St18 was cultured in a seed medium at 37° C. and 220 rpm for 12 hr to obtain the seed solution (with OD₆₀₀ being 3±0.2), and then the seed solution was inoculated into a fermentation medium containing 50 μg/mL kanamycin at an inoculation concentration of 2%. After incubation at 37° C. and 220 rpm for 3 hr, IPTG with a concentration of 0.5 mM was added. The synthesis of the phycocyanobilin was induced and carried out at 25° C. and 220 rpm for 48 hr. As shown in FIG. 4, the yield of the phycocyanobilin in the fermentation broth of the Stl8 was 15.0 mg/L.

In order to further increase the supply of heme, an endogenous gene hemEFGH (with a corresponding nucleotide sequence as shown in SEQ ID NO:6) of the E. coli was overexpressed and integrated at a yfeX site. A primer pair F12/R12 was used to amplify a gene fragment PT7-lacO-RBS-hemEFG-PT7-lacO-RBS-hemH with plasmid pET-hemEFGH (published in the paper: Xinrui Zhao, Kyeong Rok Choi, Sang Yup Lee*. Metabolic engineering of Escherichia coli for secretory production of free haem. Nature Catalysis. 2018, 1: 720-728, with a nucleotide sequence as shown in SEQ ID NO:59); Using an E. coli BL21 (DE3) genome as a template, primer pairs F13/R13 and F14/R14 were used to amplify about 500 bp homologous arms U1 and D1 upstream and downstream of the yfeX site, respectively, and the fragments were purified, Using the purified fragments U2 and D2 as templates, an integration cassette U2-PT7-lacO-RBS-hemEFG-PT7-lacO-RBS-hemH-D2 was stepwise obtained by amplification with primer pairs F15/R15 and F16/R16, and the fragment was purified. In order to obtain pTarget-yfeX for the integration at the yfeX site, the plasmid pTarget was used as a template, a primer F17/R17 was used to amplify, and the fragment was purified; and the purified fragment was transformed into E. coli JM109, the plasmid was extracted and sequenced for verification, and a correct recombinant vector pTarget-yfeX was obtained.

TABLE 3
Primer sequence

| | Sequence 5′-3′ | |
|---|---|---|
| Primer | (Underlined for the homologous arm region) | SEQ ID NO. |
| F5 | gaattttcagatgtgttttatccgggaggcattgatctcgacgctctcccttatgc | SEQ ID NO: 22 |
| R5 | ctaaacgggtcttgaggggttttttgtgagatactgatatgagcaacattac | SEQ ID NO: 23 |
| F6 | ctattaccttcctctgcacttacacattcg | SEQ ID NO: 24 |
| R6 | gatgtgttttatccgggaggcatt | SEQ ID NO: 25 |
| F7 | tgagatactgatatgagcaacattaccatttatcacaac | SEQ ID NO: 26 |
| R7 | gttatttgtaggcctgacaggcgtagcgcatcaggc | SEQ ID NO: 27 |
| F8 | gttagataagaactctctcactccctattaccttcctctgcacttacacattcg | SEQ ID NO: 28 |
| R8 | ctaaacgggtcttgaggggttttttgtgagatactgatatgagcaacattac | SEQ ID NO: 29 |
| F9 | gggtcttgaggggttttttgtgagatactgatatgagcaacattaccatttatcacaac | SEQ ID NO: 30 |
| R9 | gttatttgtaggcctgacaggcgtagcgcatcaggc | SEQ ID NO: 31 |
| F10 | aatacgggtaaagtcctgcggttttagagctagaaatagcaagttaaaataag | SEQ ID NO: 32 |
| R10 | ctctaaaaccgcaggactttacccgtattactagtattatacctaggactgagctag | SEQ ID NO: 33 |
| F11 | caatgttagccactggctaatagtattgagc | SEQ ID NO: 34 |
| R11 | ggacgcgataattggaatctcacg | SEQ ID NO: 35 |
| F12 | gatctcgacgctctcccttatgcgact | SEQ ID NO: 36 |
| R12 | tctaaacgggtcttgaggggttttttg | SEQ ID NO: 37 |
| F13 | tctaaacgggtcttgaggggttttttg | SEQ ID NO: 38 |
| R13 | cactttggtggacgacccaaac | SEQ ID NO: 39 |
| F14 | gtgtattacgcagtcaggtacgc | SEQ ID NO: 40 |
| R14 | tggcctttaatcaatgaatcagaaacgc | SEQ ID NO: 41 |
| F15 | tggcctttaatcaatgaatcagaaacgctctaaacgggtcttgaggggttttttg | SEQ ID NO: 42 |
| R15 | caatttaaacatcttagcataaagttttagagctagcactttggtggacgacccaaac | SEQ ID NO: 43 |
| F16 | ataatactagtgttagataagaactctctcactccgtgtattacgcagtcaggtacgc | SEQ ID NO: 44 |
| R16 | cactttggtggacgacccaaaccaatttaaacatcttagcataaagttttagagctag | SEQ ID NO: 45 |
| F17 | tgacggtacagaaaacccgggttttagagctagaaatagcaagttaaaataag | SEQ ID NO: 46 |
| R17 | ctctaaaacccgggttttctgtaccgtcaactagtattatacctaggactgagctag | SEQ ID NO: 47 |
| F18 | gttggcgcaggtggttttactttg | SEQ ID NO: 48 |
| R18 | caatggcgcaattatcgccg | SEQ ID NO: 49 |

### Example 4. Construction of Gene Expression Cassettes Related to Multi-Enzyme Complex of Phycocyanobilin Synthetase

A synthetic sequence of RIDD as shown in SEQ ID NO:3 was used as a template, and a primer pair F19/R19 was used for amplification. The amplified fragment was purified to obtain a fragment RIDD. A recombinant vector pRSFDuet-ho1-pcyA was used as a template, and a primer pair F20/R20 was used for amplification. Phanta MasterMix high-fidelity pfu enzyme was selected for pre-denaturation at 95° C. for 3 min. The amplification phase consisted of 30 cycles, which were carried out at 95° C. for 15 s, 58° C. for 15 s, and 72° C. for 3 min, with an extension at 72° C. for 5 min. The PCR product was purified, and a recombinant vector backbone pRSFDuet-ho1-pcyA was obtained. The fragment RIDD and the vector backbone pRSFDuet-ho1-pcyA were recombined into a recombinant vector by way of seamless cloning, and the obtained recombinant vector was transformed into E. coli JM109; and the plasmid was extracted and sequenced for verification, and a correct recombinant vector pRSFDuet-ho1-GGGGS-RIDD-pcyA was obtained.

A synthetic sequence of RIAD was used as a template to design a primer pair F21/R21, the primer pair was used for amplification to obtain a fragment RIAD, and the amplified fragment was purified. A recombinant vector pRSFDuet-ho1-GGGGS-RIDD-pcyA was used as a template to design a primer pair F22/R22, and the primer pair was used for amplification. Phanta MasterMix high-fidelity pfu enzyme was selected for pre-denaturation at 95° C. for 3 min. The amplification phase consisted of 30 cycles, which were carried out at 95° C. for 15 s, 58° C. for 15 s, and 72° C. for 3 min, with an extension at 72° C. for 5 min. The PCR product was purified, and a recombinant vector backbone pRSFDuet-ho1-pcyA was obtained. The fragment RIAD and the vector backbone pRSFDuet-ho1-GGGGS-RIDD-pcyA were recombined into a recombinant vector by way of seamless cloning, and the obtained recombinant vector was transformed into E. coli JM109; and the plasmid was extracted and sequenced for verification, and a correct recombinant vector pRSFDuet-ho1-GGGGS-RIDD-RIAD-GGGGS-pcyA was obtained. The recombinant vector pRSFDuet-ho1-GGGGS-RIDD-RIAD-GGGGS-pcyA was transformed into E. coli BL21 (DE3) ΔarsB::hemBCDAyfeX::hemEFGH to obtain an engineered strain St26. The engineered strain St26 was cultured in a seed medium at 37° C. and 220 rpm for 12 hr to obtain a seed solution (with OD₆₀₀ being 3±0.2), and then the seed solution was inoculated into a fermentation medium containing 50 μg/mL kanamycin at an inoculation concentration of 2%. After incubation at 37° C. and 220 rpm for 3 hr, IPTG with a concentration of 0.5 mM was added. The synthesis of the phycocyanobilin was induced and carried out at 25° C. and 220 rpm for 48 hr. As shown in FIG. 4, the yield of the phycocyanobilin in the fermentation broth of the St26 was 23.4 mg/L.

TABLE 4
Primer sequence

| | Sequence 5′-3′ | |
|---|---|---|
| Primer | (Underlined for the homologous arm region) | SEQ ID NO. |
| F19 | AGTCGACAAGCTTGGTGGCGGCGGCGGATCAGGTGGTGGT | SEQ ID NO: 50 |
| | GGTTCAGGTG | |
| R19 | GAACGTCTGGAAAAAGAAGAAGCCAAACGAACAGAAAGT | SEQ ID NO: 51 |
| | AATCGTATTGTACACGG | |
| F20 | TGGCGACCTCTGAAGGCTAAGTCGACAAGCTTGGTGGCGG | SEQ ID NO: 52 |
| R20 | TGGAAAAAGAAGAAGCCAAACGAACAGAAAGTAATCGTA | SEQ ID NO: 53 |
| | TTGTACACGG | |
| F21 | TTGGTGGCGGCGGCGGATCAATGGCGGTTACCGATCTGT | SEQ ID NO: 54 |
| R21 | ATTCCCCATCTTAGTATATTAGTTAAGTATAAGAAGGAGA | SEQ ID NO: 55 |
| | TATAGGTGGTGGTGGTTCAGGTGG | |
| F22 | AAGTATAAGAAGGAGATATAGGTGGTGGTGGTTCAGGTG | SEQ ID NO: 56 |
| R22 | GATTATCAAAGAAGCAACCGAAGGTTGCGTCGACAAGCTT | SEQ ID NO: 57 |
| | GGTGGCGGCGGCGGATCA | |

### Example 5. 5 L Fermentor Culture

The strain St26 constructed in Example 3 was fermented in a 5 L fermentor system.

Big and round colonies were picked and put into a 250 ml shake flask with 25 mL LB liquid medium, cultured at 37° C. and 220 rpm for 12 hr, then transferred and inoculated into a 250 mL shake flask with 25 mL LB liquid medium at an inoculation concentration of 1%, cultured at 37° C. and 220 rpm for 12 hr, then transferred and inoculated into a 5 L fermentor with 2.5 L liquid medium at an inoculation concentration of 4%, cultured at 37° C. until dissolved oxygen rebounded, and then cooled down to 25° C.; IPTG with a final concentration of 0.5 mM was added for induction, and then feeding was started; and in the fermentation process, the dissolved oxygen was controlled at 40%, the feeding flow rate was 20 ml/hr, and the pH was controlled at 7.0±0.03 by automatic dropwise addition of 50% ammonia water.

Feed medium: 50% (v/v) glycerol, 0.1 g/L FeCl₃, 6.25 g/L (NH₄)₂SO₄, and 15 g/L MgSO₄·7H₂O, with feeding flow rate being 20 mL/L.

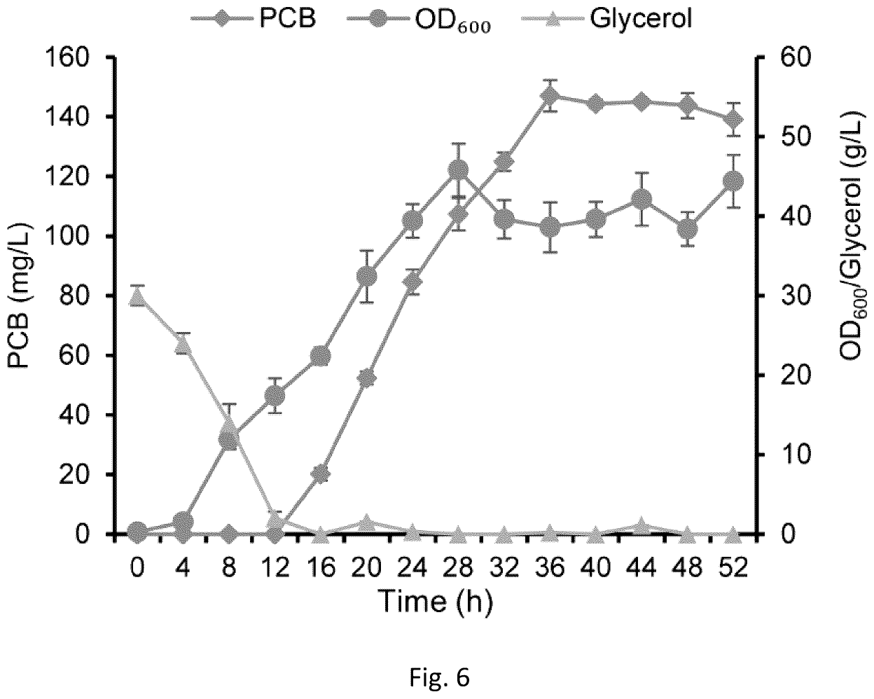

As shown in FIG. 6, after fermentation for 36 hr, the yield of the phycocyanobilin in the engineered strain can reach 147 mg/L. Compared with the initial strain, the yield of the phycocyanobilin is increased by 51.2%.

### Comparative Example 1

The implementation is the same as that in Example 2. The difference is that the pRSFDuet-1 is replaced by the plasmid pETDuet-1, and the results show that the yield of the phycocyanobilin in the constructed recombinant strain is 6.0 mg/L after fermentation for 48 hr.

### Comparative Example 2

The implementation is the same as that in Example 2. The difference is that the pRSFDuet-1 is replaced by the plasmid pCDFDuet-1, and the results show that the yield of the phycocyanobilin in the constructed recombinant strain is 5.9 mg/L after fermentation for 48 hr.

### Comparative Example 3

The implementation is the same as that in Example 2. The difference is that the pRSFDuet-1 is replaced by the plasmid pACYCDuet-1, and the results show that the yield of the phycocyanobilin in the constructed recombinant strain is 5.2 mg/L after fermentation for 48 hr.

### Comparative Example 4

The implementation is the same as that in Example 5. The difference is that the controlled fermentation condition is inoculation for 6-7 hr, and cooling induction is carried out when OD₆₀₀ is about 10. The results show that the yield of the phycocyanobilin is 74 mg/L after fermentation for 52 hr.

### Comparative Example 5

The implementation is the same as that in Example 5, except that the feed medium does not contain FeCl₃, and the results show that the yield of the phycocyanobilin is 117 mg/L after fermentation for 52 hr.

This application contains a Sequence Listing file, named WX_2023-01NP_SeqListing.xml (85 Kb) created on May 23, 2024, the content of which is incorporated by reference herein.

While the present invention has been described in some embodiments for purposes of clarity and understanding, it is not intended to limit the scope of the invention. One skilled in the art will appreciate that various changes in form and detail can be made without departing from the true scope of the invention. The true scope of the present invention shall only be as defined in the Claims.

What is claimed is:

1. A strain of recombinant Escherichia coli (E. coli) for synthesis of phycocyanobilin, wherein heme oxygenase ho1 and ferredoxin oxidoreductase pcyA derived from Synechocystis sp. PCC6803 are expressed in the strain of the recombinant E. coli.

2. The recombinant E. coli of claim 1, wherein the heme oxygenase ho1 and the ferrendoxin oxidoreductase pcyA are expressed using pRSFDuet-1 expression vector.

3. The recombinant E. coli of claim 1, wherein nucleotide sequences of genes encoding the heme oxygenase ho1 and the ferredoxin oxidoreductase pcyA are shown in SEQ ID NO:1 and SEQ ID NO:2, respectively.

4. The recombinant E. coli of claim 1, wherein the heme oxygenase ho1 and the ferredoxin oxidoreductase pcyA are linked together by short peptide tags RIDD and RIAD.

5. The recombinant E. coli of claim 1, wherein at least one of the following improvements is made in the recombinant E. coli:
(1) overexpression of endogenous genes hemB, hemC, and hemD; and
(2) overexpression of endogenous genes hemE, hemF, hemG, and hemH.

6. The recombinant E. coli of claim 5, wherein the genes hemB, hemC, and hemD are integrated at an arsB site of an arsenate transporter gene of the recombinant E. coli.

7. The recombinant E. coli of claim 5, wherein the genes hemE, hemF, hemG and hemH are integrated at a position where a heme degrading gene yfeX is located.

8. The recombinant E. coli of claim 4, a fusion gene ho1-GGGGS-RIDD-RIAD-GGGGS-pcyA is expressed in E. coli strain BL21(DE3) using pRSFDuet-1 as the expression vector, wherein endogenous genes hemB, hemC, hemD, hemE, hemF, hemG, and hemH are overexpressed; wherein the nucleotide sequence of the fusion gene ho1-GGGGS-RIDD-RIAD-GGGGS-pcyA is shown in SEQ ID NO:60.

9. The recombinant E. coli of claim 7, wherein the genes hemB, hemC and hemD are integrated at an arsB site of an arsenate transporter gene of the recombinant E. coli.

10. The recombinant E. coli according to claim 8, wherein the genes hemE, hemF, hemG, and hemH are integrated at a position where a heme degrading gene yfeX is located.

11. A method for producing phycocyanobilin, wherein the recombinant E. coli of claim 1 is fermented to produce the phycocyanobilin.

12. The method of claim 11, wherein the recombinant E. coli is inoculated into a fermentation medium, cultured at 35-37° C. for 2-3 hr, induced with IPTG, and fermented for 24-48 hr.

13. The method of claim 12, wherein 0.5 mM IPTG is added and synthesis of phycocyanobilin is induced and carried out at 25° C., 200-220 rpm for 24-48 hr.

14. The method of claim 12, wherein the fermentation medium contains KH₂PO₄, K₂HPO₄·3H₂O, (NH₄)₂SO₄, anhydrous citric acid, MgSO₄, yeast powder, glycerol, maltodextrin, vitamin B1, and a trace element solution.

15. The method of claim 14, wherein the trace element solution contains Fe(III) citrate, ZnCl₂, MnSO₄·H₂O, CuSO₄·5H₂O, Na₂MoO₄·2H₂O, CaCl₂·2H₂O, H₃BO₃, CoCl₂·6H₂O, and NiSO₄·6H₂O.


SEQUENCE LISTING (60 sequences)

SEQ ID NO: 1 - DNA, 723 bp, synthetic construct
  source 1..723: mol_type = other DNA
atgagcgttaacctggcgagccagctgcgtgaaggcaccaaaaaatctcactctatggcggaaaacgttggtttcgttaaatgcttcctgaaaggtgttgttgagaaaaacagctaccgtaaactggttggtaacctgtacttcgtttacagcgcgatggaagaagaaatggcgaaattcaaagatcacccgatcctgtctcacatctacttcccggaactgaaccgtaaacagtctctggaacaggatctgcagttctactatggtagcaactggcgtcaggaagttaaaatctctgcggcgggtcaggcgtacgttgatcgcgttcgtcaggttgcggcgaccgcgccggaactgctggttgcgcacagctacacccgttacctgggcgatctgagcggcggccagatcctgaagaaaatcgcgcagaacgcgatgaacctgcacgatggtggcaccgcgttctacgaattcgcagatatcgatgatgaaaaagcgttcaaaaacacctaccgtcaggctatgaacgatctgccgatcgatcaggcgaccgcggaacgtatcgttgatgaagcgaacgatgcgttcgcgatgaacatgaaaatgttcaacgaactggaaggtaacctgatcaaagcgatcggcatcatggttttcaactctctgacccgtcgtcgtagccagggcagcaccgaagttggtctggcgacctctgaaggctaa

SEQ ID NO: 2 - DNA, 747 bp, synthetic construct
  source 1..747: mol_type = other DNA
atggcggttaccgatctgtctctgaccaacagcagcctgatgccgaccctgaacccgatgatccagcagctggcgctggcgatcgcggcgtcttggcagagcctgccgctgaaaccgtaccagctgccggaagatctgggttacgttgaaggccgtctggaaggtgaaaaactggttatcgaaaaccgttgctaccagaccccgcagttccgtaaaatgcacctggaactggcaaaagtgggcaaaggtctggatatcctgcactgcgttatgttcccggaaccgctgtacggcctgccgctgttcggttgcgatatcgttgcgggtccgggtggtgttagcgcggctatcgcggatctgtctccgacccagtctgatcgtcagctgccggcggcgtaccagaaatctctggcggaactgggtcagccggaattcgaacagcagcgtgaactgccgccgtggggtgaaatcttctctgaatactgcctgttcattcgtccgtctaacgttaccgaagaagaacgtttcgttcagcgtgttgttgatttcctgcagatccactgccaccagagcatcgtggcggaaccgctgagcgaagcgcagaccctggaacaccgtcagggccagatccactactgccagcagcagcagaaaaacgataaaacccgtcgtgttctggaaaaagcgttcggcgaagcgtgggcggaacgttacatgtctcaggttctgttcgatgttatccagtaa

SEQ ID NO: 3 - DNA, 198 bp, synthetic construct
  source 1..198: mol_type = other DNA
ggtggtggtggttcaggtggtggtggttcaggtggtggtggttgtggtagcctgcgtgaatgtgaactgtatgttcagaaacataatattcaggccctgctgaaagatagcattgttcagctgtgtaccgcacgtccggaacgtccgatggcatttctgcgcgaatattttgaacgtctggaaaaagaagaagccaaa

SEQ ID NO: 4 - DNA, 108 bp, synthetic construct
  source 1..108: mol_type = other DNA
ggtggtggtggttcaggtggtggtggttcaggtggtggtggttgtggtctggaacagtatgcaaatcagctggcagatcagattatcaaagaagcaaccgaaggttgc

SEQ ID NO: 5 - DNA, 3122 bp, synthetic construct
  source 1..3122: mol_type = other DNA
taatacgactcactataggggaattgtgagcggataacaattcccctgtagaaataattttgtttaactttaataaggagatataccatgggtacagacttaatccaacgccctcgtcgcctgcgcaaatctcctgcgctgcgcgctatgtttgaagagacaacacttagccttaacgacctggtgttgccgatctttgttgaagaagaaattgacgactacaaagccgttgaagccatgccaggtgtgatgcgcattccagagaaacatctggcacgcgaaattgaacgcatcgccaacgccggtattcgttccgtgatgactttcggcatctctcaccataccgatgaaaccggcagcgatgcctggcgggaagatggactggtggcgcgaatgtcgcgcatctgcaagcagaccgtgccagaaatgatcgtcatgtcagacacctgcttctgcgaatacacatctcacggtcactgcggtgtgctgtgcgagcatggcgtcgacaacgacgcgactctggaaaatttaggcaagcaagccgtggttgcagctgctgcaggcgcagacttcatcgccccttctgccgcgatggacggccaggtacaggcgattcgccaggcgctggacgctgcgggctttaaagatacggcgattatgtcgtattcgaccaagttcgcctcttccttttatggtccgttccgtgaagctgccggaagcgcattaaaaggcgaccgcaaaagctatcagatgaacccaatgaaccgtcgtgaggcgattcgtgagtcactgctggatgaagcccagggcgcagactgtctgatggttaaacctgccggagcgtacctcgacatcgtgcgtgagctgcgtgaacgtactgaattgccgattggcgcgtatcaggtgagcggtgagtacgcgatgattaagttcgccgcgctggcgggtgctatagatgaagagaaagtcgtgctcgaaagcttaggttcaattaagcgtgcgggtgcggatctgattttcagctactttgcgatggatttggctgagaagaagattctgcgttaaggatccgaattcgagctcggcgcgcctgcaggtcgacaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgttagacaatgttttaagaattgccacacgccaaagcccacttgcactctggcaggcacactatgtcaaagacaagttgatggcgagccatccgggcctggtcgttgaactggtaccgatggtgacgcgcggcgatgtgattcttgatacgccgctggcgaaagtaggcggaaaaggcttatttgttaaagagctggaagtcgcgctcctcgaaaatcgcgccgatatcgccgtacattcaatgaaagatgtgccggttgaattcccgcaaggtctgggactggtcactatttgtgagcgtgaagatcctcgcgatgcctttgtgtccaataactatgacaatctggatgcgttaccggcaggcagtatcgtcgggacgtccagtttacgtcgccagtgccaactggctgaacgccgcccggatctgattatccgctccctgcgaggcaacgtcggcactcgcctgagtaaactggataacggcgaatacgatgccatcattcttgcggtagccggactaaaacgtttaggtctggagtcccgcattcgcgccgcattgccacccgagatttctcttccggcggtaggacaaggtgcggtgggtattgaatgccgccttgatgattctcgcactcgcgagctgcttgccgcgctgaatcaccacgaaactgcactgcgcgttaccgcagaacgcgccatgaatacccgtctcgaaggcggatgtcaggtgccaattggtagctacgccgagcttattgatggcgaaatctggctgcgtgcgttggtcggcgcgccggacggttcgcagattattcgcggtgaacgccgcggtgcgccgcaagatgccgaacaaatggggatttcgctggcagaagagctactgaataacggcgcgcgcgagatcctcgctgaagtctataacggagacgctccggcatgaagatctataaaaggaggaaaatatatgagtatcctggtcacccgcccgtctcccgctggagaagagttagtgagccgtctgcgcacactggggcaggtggcctggcattttccactgattgagttttctccgggtcgacaattaccacaacttgctgatcaactggcggcgctgggggagagcgatctgttgtttgccctctcgcaacacgcggttgcttttgcccaatcacagctgcatcagcaagatcgtaaatggccccgactacctgattatttcgccattggacgcaccaccgcactggcactacataccgtaagcggacagaagattctctacccgcaggatcgggaaatcagcgaagtcttgctacaattacctgaattacaaaatattgcgggcaaacgtgcgctgatattacgtggcaatggcggtcgtgagctaattggggataccctgacggcgcgcggtgctgaggtcactttttgtgaatgttatcaacgatgcgcaatccattacgatggtgcagaagaagcgatgcgctggcaatcccgcgaggtgacgacggtcgttgttaccagcggtgaaatgttgcagcaactctggtcgctgatcccacaatggtatcgtgagcactggttactacactgtcgactattggtcgtcagtgagcgtttggcgaaactcgcccgggaactgggctggcaagacattaaggtcgccgataacgctgacaacgatgcgcttttacgggcattacaataactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttg

SEQ ID NO: 6 - DNA, 4228 bp, synthetic construct
  source 1..4228: mol_type = other DNA
gatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatataccatgggtaccgaacttaaaaacgatcgttatctgcgggcgctgctgcgccagcccgttgatgtcactccagtatggatgatgcgccaggcgggtcgctatctaccggaatataaagccacgcgcgcccaggcgggcgattttatgtcgctgtgcaaaaacgccgagctggcgtgcgaagtgactttgcaaccgctgcgtcgctacccgctggatgcggcgatcctcttttccgatatcctcaccgtgccggacgcgatggggttagggctctattttgaagccggagaaggtccgcgttttacctcgccagtcacctgcaaagccgacgtcgataaactgccaattccggacccggaagatgagctgggttacgtgatgaacgcggtgcgtaccattcgtcgcgaactgaaaggcgaagtgccgctgattggtttttccggcagcccgtggacgctggcgacctacatggtggaaggcggcagcagcaaagcgttcaccgtgatcaaaaaaatgatgtatgccgatccgcaggcgctgcacgctctactcgataaactggcgaaaagcgtcactttgtatctgaatgcgcagattaaagccggtgctcaggcagtgatgattttcgacacctggggcggtgtgcttaccgggcgcgattatcaacagttctcgctctattacatgcataaaattgttgatggtttactgcgtgaaaacgacggtcgccgcgtaccggtcacgctgtttaccaaaggcggcggacagtggctggaagcgatggcagaaaccggttgcgatgcgttgggcctcgactggacaacggatatcgccgatgcgcgccgccgtgtgggcaataaagtcgcgttgcagggtaatatggatccgtcgatgctgtacgctccgcctgcccgcattgaagaagaagtagcgactatacttgcaggtttcggtcacggcgaaggtcatgtctttaaccttggtcacggcattcatcaggatgtgccgccagaacatgctggcgtattcgtggaggcagtgcatcgactgtctgaacaatatcaccgctaagagctcataaaaggaggaaaatatatgaaacccgacgcacaccaggttaaacagtttctgctcaaccttcaggatacgatttgtcagcagctgaccgccgtcgatggcgcagaatttgtcgaagatagttggcagcgcgaagctggcggcggcgggcgtagtcgggtgttgcgtaatggtggtgttttcgaacaggcaggcgtcaacttttcgcatgtccacggtgaggcgatgcctgcttccgccaccgctcatcgcccggaacttgccgggcgcagtttcgaggcgatgggcgtttcactggtagtgcatccgcataacccgtatgttcccaccagccacgcgaatgtgcggttttttattgccgaaaaaccgggtgccgatcccgtctggtggtttggcggcggcttcgatttaacccctttctatggttttgaagaagacgccattcactggcaccgcaccgcccgtgacctgtgcctgccatttggtgaagacgtttatccccgttacaaaaagtggtgcgacgattacttctacctcaaacatcgcaacgaacagcgcggtattggcgggctgttctttgatgatctgaacacgccagatttcgaccactgttttgcctttatgcaggcggtaggcaaaggctacaccgacgcttatttaccaattgtagagcgacgtaaagcgatggcctacggcgagcgcgagcgcaattttcagctctaccgtcgcggtcgttatgtcgagttcaatctggtctgggatcgcggcacgctgtttggcctgcaaactggcgggcgcaccgagtctatcctgatgtcaatgccgccactggtacgctgggaatatgattatcagccaaaagatggcagcccagaagcggcgttaagtgagtttattaaggtcagggattgggtgtaactgcagataaaaggaggaaaatatgtgaaaacattaattcttttctcaacaagggacggacaaacgcgcgagattgcctcctacctggcttcggaactgaaagaactggggatccaggcggatgtcgccaatgtgcaccgcattgaagaaccacagtgggaaaactatgaccgtgtggtcattggtgcttctattcgctatggtcactaccattcagcgttccaggaatttgtcaaaaaacatgcgacgcggctgaattcgatgccgagcgccttttactccgtgaatctggtggcgcgcaaaccggagaagcgtactccacagaccaacagctacgcgcgcaagtttctgatgaactcgcaatggcgtcccgatcgctgcgcggtcattgccggggcgctgcgttacccacgttatcgctggtacgaccgttttatgatcaagctgattatgaagatgtcaggcggtgaaacggatacgcgcaaagaagttgtctataccgattgggagcaggtggcgaatttcgcccgagaaatcgcccatttaaccgacaaaccgacgctgaaataagcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgcgtcagactaaaaccggtatcctgctggcaaacctgggtacgcccgatgcccccacacctgaagcggtaaaacgctatctgaaacaatttttaagcgacagacgcgtggttgatacctcacggttgttatggtggccattgctgcgcggcgtgattttgccgctgcgctcgccgcgtgtggcgaagctgtatgcctctgtctggatggaaggtggctcgccgctgatggtttacagccgtcagcaacagcaggcgctggcacaacgtttaccggagacgcccgtagcgctgggaatgagctacggctcgccatcactggaaagcgccgtagatgaactcctggcagagcatgtagatcatattgtggtgctgccgctttatccgcaatactcctgttcaacggtcggtgcggtatgggatgaactggcacgcattctggcgcgcaaacgtagcattccggggatatcgtttattcgtgattacgctgataaccacgattacattaatgcactggcgaacagcgtacgcgcttcttttgccaaacatggcgaaccggatctgctgctgctctcttatcatggcattccccagcgttatgcagatgaaggcgatgattacccgcaacgttgccgcacaacgactcgcgaactggcttccgcactggggatggcaccggaaaaagtgatgatgacctttcagtcgcgctttggtcgggaaccctggctgatgccttataccgacgaaacgctgaaaatgctcggagaaaaaggcgtaggtcatatacaggtgatgtgcccgggctttgctgcggattgtctggagacgctggaagagattgccgagcaaaaccgtgaggtcttcctcggtgccggcgggaaaaaatatgaatatattccagcgcttaatgccacgccggaacatattgaaatgatggctaatcttgttgccgcgtatcgctaactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttg

SEQ ID NO: 7 - DNA, 972 bp, synthetic construct
  source 1..972: mol_type = other DNA
acagacttaatccaacgccctcgtcgcctgcgcaaatctcctgcgctgcgcgctatgtttgaagagacaacacttagccttaacgacctggtgttgccgatctttgttgaagaagaaattgacgactacaaagccgttgaagccatgccaggtgtgatgcgcattccagagaaacatctggcacgcgaaattgaacgcatcgccaacgccggtattcgttccgtgatgactttcggcatctctcaccataccgatgaaaccggcagcgatgcctggcgggaagatggactggtggcgcgaatgtcgcgcatctgcaagcagaccgtgccagaaatgatcgtcatgtcagacacctgcttctgcgaatacacatctcacggtcactgcggtgtgctgtgcgagcatggcgtcgacaacgacgcgactctggaaaatttaggcaagcaagccgtggttgcagctgctgcaggcgcagacttcatcgccccttctgccgcgatggacggccaggtacaggcgattcgccaggcgctggacgctgcgggctttaaagatacggcgattatgtcgtattcgaccaagttcgcctcttccttttatggtccgttccgtgaagctgccggaagcgcattaaaaggcgaccgcaaaagctatcagatgaacccaatgaaccgtcgtgaggcgattcgtgagtcactgctggatgaagcccagggcgcagactgtctgatggttaaacctgccggagcgtacctcgacatcgtgcgtgagctgcgtgaacgtactgaattgccgattggcgcgtatcaggtgagcggtgagtacgcgatgattaagttcgccgcgctggcgggtgctatagatgaagagaaagtcgtgctcgaaagcttaggttcaattaagcgtgcgggtgcggatctgattttcagctactttgcgatggatttggctgagaagaagattctgcgttaa

SEQ ID NO: 8 - DNA, 939 bp, synthetic construct
  source 1..939: mol_type = other DNA
ttagacaatgttttaagaattgccacacgccaaagcccacttgcactctggcaggcacactatgtcaaagacaagttgatggcgagccatccgggcctggtcgttgaactggtaccgatggtgacgcgcggcgatgtgattcttgatacgccgctggcgaaagtaggcggaaaaggcttatttgttaaagagctggaagtcgcgctcctcgaaaatcgcgccgatatcgccgtacattcaatgaaagatgtgccggttgaattcccgcaaggtctgggactggtcactatttgtgagcgtgaagatcctcgcgatgcctttgtgtccaataactatgacaatctggatgcgttaccggcaggcagtatcgtcgggacgtccagtttacgtcgccagtgccaactggctgaacgccgcccggatctgattatccgctccctgcgaggcaacgtcggcactcgcctgagtaaactggataacggcgaatacgatgccatcattcttgcggtagccggactaaaacgtttaggtctggagtcccgcattcgcgccgcattgccacccgagatttctcttccggcggtaggacaaggtgcggtgggtattgaatgccgccttgatgattctcgcactcgcgagctgcttgccgcgctgaatcaccacgaaactgcactgcgcgttaccgcagaacgcgccatgaatacccgtctcgaaggcggatgtcaggtgccaattggtagctacgccgagcttattgatggcgaaatctggctgcgtgcgttggtcggcgcgccggacggttcgcagattattcgcggtgaacgccgcggtgcgccgcaagatgccgaacaaatggggatttcgctggcagaagagctactgaataacggcgcgcgcgagatcctcgctgaagtctataacggagacgctccggcatga

SEQ ID NO: 9 - DNA, 741 bp, synthetic construct
  source 1..741: mol_type = other DNA
atgagtatcctggtcacccgcccgtctcccgctggagaagagttagtgagccgtctgcgcacactggggcaggtggcctggcattttccactgattgagttttctccgggtcgacaattaccacaacttgctgatcaactggcggcgctgggggagagcgatctgttgtttgccctctcgcaacacgcggttgcttttgcccaatcacagctgcatcagcaagatcgtaaatggccccgactacctgattatttcgccattggacgcaccaccgcactggcactacataccgtaagcggacagaagattctctacccgcaggatcgggaaatcagcgaagtcttgctacaattacctgaattacaaaatattgcgggcaaacgtgcgctgatattacgtggcaatggcggtcgtgagctaattggggataccctgacggcgcgcggtgctgaggtcactttttgtgaatgttatcaacgatgcgcaatccattacgatggtgcagaagaagcgatgcgctggcaatcccgcgaggtgacgacggtcgttgttaccagcggtgaaatgttgcagcaactctggtcgctgatcccacaatggtatcgtgagcactggttactacactgtcgactattggtcgtcagtgagcgtttggcgaaactcgcccgggaactgggctggcaagacattaaggtcgccgataacgctgacaacgatgcgcttttacgggcattacaataa

SEQ ID NO: 10 - DNA, 1062 bp, synthetic construct
  source 1..1062: mol_type = other DNA
accgaacttaaaaacgatcgttatctgcgggcgctgctgcgccagcccgttgatgtcactccagtatggatgatgcgccaggcgggtcgctatctaccggaatataaagccacgcgcgcccaggcgggcgattttatgtcgctgtgcaaaaacgccgagctggcgtgcgaagtgactttgcaaccgctgcgtcgctacccgctggatgcggcgatcctcttttccgatatcctcaccgtgccggacgcgatggggttagggctctattttgaagccggagaaggtccgcgttttacctcgccagtcacctgcaaagccgacgtcgataaactgccaattccggacccggaagatgagctgggttacgtgatgaacgcggtgcgtaccattcgtcgcgaactgaaaggcgaagtgccgctgattggtttttccggcagcccgtggacgctggcgacctacatggtggaaggcggcagcagcaaagcgttcaccgtgatcaaaaaaatgatgtatgccgatccgcaggcgctgcacgctctactcgataaactggcgaaaagcgtcactttgtatctgaatgcgcagattaaagccggtgctcaggcagtgatgattttcgacacctggggcggtgtgcttaccgggcgcgattatcaacagttctcgctctattacatgcataaaattgttgatggtttactgcgtgaaaacgacggtcgccgcgtaccggtcacgctgtttaccaaaggcggcggacagtggctggaagcgatggcagaaaccggttgcgatgcgttgggcctcgactggacaacggatatcgccgatgcgcgccgccgtgtgggcaataaagtcgcgttgcagggtaatatggatccgtcgatgctgtacgctccgcctgcccgcattgaagaagaagtagcgactatacttgcaggtttcggtcacggcgaaggtcatgtctttaaccttggtcacggcattcatcaggatgtgccgccagaacatgctggcgtattcgtggaggcagtgcatcgactgtctgaacaatatcaccgctaa

SEQ ID NO: 11 - DNA, 900 bp, synthetic construct
  source 1..900: mol_type = other DNA
atgaaacccgacgcacaccaggttaaacagtttctgctcaaccttcaggatacgatttgtcagcagctgaccgccgtcgatggcgcagaatttgtcgaagatagttggcagcgcgaagctggcggcggcgggcgtagtcgggtgttgcgtaatggtggtgttttcgaacaggcaggcgtcaacttttcgcatgtccacggtgaggcgatgcctgcttccgccaccgctcatcgcccggaacttgccgggcgcagtttcgaggcgatgggcgtttcactggtagtgcatccgcataacccgtatgttcccaccagccacgcgaatgtgcggttttttattgccgaaaaaccgggtgccgatcccgtctggtggtttggcggcggcttcgatttaacccctttctatggttttgaagaagacgccattcactggcaccgcaccgcccgtgacctgtgcctgccatttggtgaagacgtttatccccgttacaaaaagtggtgcgacgattacttctacctcaaacatcgcaacgaacagcgcggtattggcgggctgttctttgatgatctgaacacgccagatttcgaccactgttttgcctttatgcaggcggtaggcaaaggctacaccgacgcttatttaccaattgtagagcgacgtaaagcgatggcctacggcgagcgcgagcgcaattttcagctctaccgtcgcggtcgttatgtcgagttcaatctggtctgggatcgcggcacgctgtttggcctgcaaactggcgggcgcaccgagtctatcctgatgtcaatgccgccactggtacgctgggaatatgattatcagccaaaagatggcagcccagaagcggcgttaagtgagtttattaaggtcagggattgggtgtaa

SEQ ID NO: 12 - DNA, 546 bp, synthetic construct
  source 1..546: mol_type = other DNA
gtgaaaacattaattcttttctcaacaagggacggacaaacgcgcgagattgcctcctacctggcttcggaactgaaagaactggggatccaggcggatgtcgccaatgtgcaccgcattgaagaaccacagtgggaaaactatgaccgtgtggtcattggtgcttctattcgctatggtcactaccattcagcgttccaggaatttgtcaaaaaacatgcgacgcggctgaattcgatgccgagcgccttttactccgtgaatctggtggcgcgcaaaccggagaagcgtactccacagaccaacagctacgcgcgcaagtttctgatgaactcgcaatggcgtcccgatcgctgcgcggtcattgccggggcgctgcgttacccacgttatcgctggtacgaccgttttatgatcaagctgattatgaagatgtcaggcggtgaaacggatacgcgcaaagaagttgtctataccgattgggagcaggtggcgaatttcgcccgagaaatcgcccatttaaccgacaaaccgacgctgaaataa

SEQ ID NO: 13 - DNA, 960 bp, synthetic construct
  source 1..960: mol_type = other DNA
cgtcagactaaaaccggtatcctgctggcaaacctgggtacgcccgatgcccccacacctgaagcggtaaaacgctatctgaaacaatttttaagcgacagacgcgtggttgatacctcacggttgttatggtggccattgctgcgcggcgtgattttgccgctgcgctcgccgcgtgtggcgaagctgtatgcctctgtctggatggaaggtggctcgccgctgatggtttacagccgtcagcaacagcaggcgctggcacaacgtttaccggagacgcccgtagcgctgggaatgagctacggctcgccatcactggaaagcgccgtagatgaactcctggcagagcatgtagatcatattgtggtgctgccgctttatccgcaatactcctgttcaacggtcggtgcggtatgggatgaactggcacgcattctggcgcgcaaacgtagcattccggggatatcgtttattcgtgattacgctgataaccacgattacattaatgcactggcgaacagcgtacgcgcttcttttgccaaacatggcgaaccggatctgctgctgctctcttatcatggcattccccagcgttatgcagatgaaggcgatgattacccgcaacgttgccgcacaacgactcgcgaactggcttccgcactggggatggcaccggaaaaagtgatgatgacctttcagtcgcgctttggtcgggaaccctggctgatgccttataccgacgaaacgctgaaaatgctcggagaaaaaggcgtaggtcatatacaggtgatgtgcccgggctttgctgcggattgtctggagacgctggaagagattgccgagcaaaaccgtgaggtcttcctcggtgccggcgggaaaaaatatgaatatattccagcgcttaatgccacgccggaacatattgaaatgatggctaatcttgttgccgcgtatcgctaa

SEQ ID NO: 14 - DNA, 35 bp, synthetic construct
  source 1..35: mol_type = other DNA
taaggagatataatgagcgttaacctggcgagcca

SEQ ID NO: 15 - DNA, 50 bp, synthetic construct
  source 1..50: mol_type = other DNA
ttggtctggcgacctctgaaggctaacgaacagaaagtaatcgtattgta

SEQ ID NO: 16 - DNA, 23 bp, synthetic construct
  source 1..23: mol_type = other DNA
cgaacagaaagtaatcgtattgt

SEQ ID NO: 17 - DNA, 44 bp, synthetic construct
  source 1..44: mol_type = other DNA
aggttaacgctcattatatctccttattaaagttaaacaaaatt

SEQ ID NO: 18 - DNA, 30 bp, synthetic construct
  source 1..30: mol_type = other DNA
atataatggcggttaccgatctgtctctga

SEQ ID NO: 19 - DNA, 49 bp, synthetic construct
  source 1..49: mol_type = other DNA
gttctgttcgatgttatccagtaagcagatctcaattggatatcggccg

SEQ ID NO: 20 - DNA, 47 bp, synthetic construct
  source 1..47: mol_type = other DNA
attagttaagtataagaaggagatataatggcggttaccgatctgtc

SEQ ID NO: 21 - DNA, 25 bp, synthetic construct
  source 1..25: mol_type = other DNA
gcagatctcaattggatatcggccg

SEQ ID NO: 22 - DNA, 56 bp, synthetic construct
  source 1..56: mol_type = other DNA
gaattttcagatgtgttttatccgggaggcattgatctcgacgctctcccttatgc

SEQ ID NO: 23 - DNA, 52 bp, synthetic construct
  source 1..52: mol_type = other DNA
ctaaacgggtcttgaggggttttttgtgagatactgatatgagcaacattac

SEQ ID NO: 24 - DNA, 30 bp, synthetic construct
  source 1..30: mol_type = other DNA
ctattaccttcctctgcacttacacattcg

SEQ ID NO: 25 - DNA, 24 bp, synthetic construct
  source 1..24: mol_type = other DNA
gatgtgttttatccgggaggcatt

SEQ ID NO: 26 - DNA, 39 bp, synthetic construct
  source 1..39: mol_type = other DNA
tgagatactgatatgagcaacattaccatttatcacaac

SEQ ID NO: 27 - DNA, 36 bp, synthetic construct
  source 1..36: mol_type = other DNA
gttatttgtaggcctgacaggcgtagcgcatcaggc

SEQ ID NO: 28 - DNA, 54 bp, synthetic construct
  source 1..54: mol_type = other DNA
gttagataagaactctctcactccctattaccttcctctgcacttacacattcg

SEQ ID NO: 29 - DNA, 52 bp, synthetic construct; identical to SEQ ID NO: 23
  source 1..52: mol_type = other DNA

SEQ ID NO: 30 - DNA, 59 bp, synthetic construct
  source 1..59: mol_type = other DNA
gggtcttgaggggttttttgtgagatactgatatgagcaacattaccatttatcacaac

SEQ ID NO: 31 - DNA, 36 bp, synthetic construct; identical to SEQ ID NO: 27
  source 1..36: mol_type = other DNA

SEQ ID NO: 32 - DNA, 53 bp, synthetic construct
  source 1..53: mol_type = other DNA
aatacgggtaaagtcctgcggttttagagctagaaatagcaagttaaaataag

SEQ ID NO: 33 - DNA, 57 bp, synthetic construct
  source 1..57: mol_type = other DNA
ctctaaaaccgcaggactttacccgtattactagtattatacctaggactgagctag

SEQ ID NO: 34 - DNA, 31 bp, synthetic construct
  source 1..31: mol_type = other DNA
caatgttagccactggctaatagtattgagc

SEQ ID NO: 35 - DNA, 24 bp, synthetic construct
  source 1..24: mol_type = other DNA
ggacgcgataattggaatctcacg

SEQ ID NO: 36 - DNA, 27 bp, synthetic construct
  source 1..27: mol_type = other DNA
gatctcgacgctctcccttatgcgact

SEQ ID NO: 37 - DNA, 27 bp, synthetic construct
  source 1..27: mol_type = other DNA
tctaaacgggtcttgaggggttttttg

SEQ ID NO: 38 - DNA, 27 bp, synthetic construct
  source 1..27: mol_type = other DNA
tctaaacgggtcttgaggggttttttg

SEQ ID NO: 39 - DNA, 22 bp, synthetic construct
  source 1..22: mol_type = other DNA
cactttggtggacgacccaaac

SEQ ID NO: 40 - DNA, 23 bp, synthetic construct
  source 1..23: mol_type = other DNA
gtgtattacgcagtcaggtacgc

SEQ ID NO: 41 - DNA, 28 bp, synthetic construct
  source 1..28: mol_type = other DNA
tggcctttaatcaatgaatcagaaacgc

SEQ ID NO: 42 - DNA, 55 bp, synthetic construct
  source 1..55: mol_type = other DNA
tggcctttaatcaatgaatcagaaacgctctaaacgggtcttgaggggttttttg

SEQ ID NO: 43 - DNA, 58 bp, synthetic construct
  source 1..58: mol_type = other DNA
caatttaaacatcttagcataaagttttagagctagcactttggtggacgacccaaac

SEQ ID NO: 44 - DNA, 58 bp, synthetic construct
  source 1..58: mol_type = other DNA
ataatactagtgttagataagaactctctcactccgtgtattacgcagtcaggtacgc

SEQ ID NO: 45 - DNA, 58 bp, synthetic construct
  source 1..58: mol_type = other DNA
cactttggtggacgacccaaaccaatttaaacatcttagcataaagttttagagctag

SEQ ID NO: 46 - DNA, 53 bp, synthetic construct
  source 1..53: mol_type = other DNA
tgacggtacagaaaacccgggttttagagctagaaatagcaagttaaaataag

SEQ ID NO: 47 - DNA, 57 bp, synthetic construct
  source 1..57: mol_type = other DNA
ctctaaaacccgggttttctgtaccgtcaactagtattatacctaggactgagctag

SEQ ID NO: 48 - DNA, 24 bp, synthetic construct
  source 1..24: mol_type = other DNA
gttggcgcaggtggttttactttg

SEQ ID NO: 49 - DNA, 20 bp, synthetic construct
  source 1..20: mol_type = other DNA
caatggcgcaattatcgccg

SEQ ID NO: 50 - DNA, 50 bp, synthetic construct
  source 1..50: mol_type = other DNA
agtcgacaagcttggtggcggcggcggatcaggtggtggtggttcaggtg

SEQ ID NO: 51 - DNA, 56 bp, synthetic construct
  source 1..56: mol_type = other DNA
gaacgtctggaaaaagaagaagccaaacgaacagaaagtaatcgtattgtacacgg

SEQ ID NO: 52 - DNA, 40 bp, synthetic construct
  source 1..40: mol_type = other DNA
tggcgacctctgaaggctaagtcgacaagcttggtggcgg

SEQ ID NO: 53 - DNA, 49 bp, synthetic construct
  source 1..49: mol_type = other DNA
tggaaaaagaagaagccaaacgaacagaaagtaatcgtattgtacacgg

SEQ ID NO: 54 - DNA, 39 bp, synthetic construct
  source 1..39: mol_type = other DNA
ttggtggcggcggcggatcaatggcggttaccgatctgt

SEQ ID NO: 55 - DNA, 64 bp, synthetic construct
  source 1..64: mol_type = other DNA
attccccatcttagtatattagttaagtataagaaggagatataggtggtggtggttcaggtgg

SEQ ID NO: 56 - DNA, 39 bp, synthetic construct
  source 1..39: mol_type = other DNA
aagtataagaaggagatataggtggtggtggttcaggtg

SEQ ID NO: 57 - DNA, 58 bp, synthetic construct
  source 1..58: mol_type = other DNA
gattatcaaagaagcaaccgaaggttgcgtcgacaagcttggtggcggcggcggatca

SEQ ID NO: 58 - DNA, 6425 bp, synthetic construct
  source 1..6425: mol_type = other DNA
ggggaattgtgagcggataacaattcccctgtagaaataattttgtttaactttaataaggagatataccatgggtacagacttaatccaacgccctcgtcgcctgcgcaaatctcctgcgctgcgcgctatgtttgaagagacaacacttagccttaacgacctggtgttgccgatctttgttgaagaagaaattgacgactacaaagccgttgaagccatgccaggtgtgatgcgcattccagagaaacatctggcacgcgaaattgaacgcatcgccaacgccggtattcgttccgtgatgactttcggcatctctcaccataccgatgaaaccggcagcgatgcctggcgggaagatggactggtggcgcgaatgtcgcgcatctgcaagcagaccgtgccagaaatgatcgtcatgtcagacacctgcttctgcgaatacacatctcacggtcactgcggtgtgctgtgcgagcatggcgtcgacaacgacgcgactctggaaaatttaggcaagcaagccgtggttgcagctgctgcaggcgcagacttcatcgccccttctgccgcgatggacggccaggtacaggcgattcgccaggcgctggacgctgcgggctttaaagatacggcgattatgtcgtattcgaccaagttcgcctcttccttttatggtccgttccgtgaagctgccggaagcgcattaaaaggcgaccgcaaaagctatcagatgaacccaatgaaccgtcgtgaggcgattcgtgagtcactgctggatgaagcccagggcgcagactgtctgatggttaaacctgccggagcgtacctcgacatcgtgcgtgagctgcgtgaacgtactgaattgccgattggcgcgtatcaggtgagcggtgagtacgcgatgattaagttcgccgcgctggcgggtgctatagatgaagagaaagtcgtgctcgaaagcttaggttcaattaagcgtgcgggtgcggatctgattttcagctactttgcgatggatttggctgagaagaagattctgcgttaaggatccgaattcgagctcggcgcgcctgcaggtcgacaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgttagacaatgttttaagaattgccacacgccaaagcccacttgcactctggcaggcacactatgtcaaagacaagttgatggcgagccatccgggcctggtcgttgaactggtaccgatggtgacgcgcggcgatgtgattcttgatacgccgctggcgaaagtaggcggaaaaggcttatttgttaaagagctggaagtcgcgctcctcgaaaatcgcgccgatatcgccgtacattcaatgaaagatgtgccggttgaattcccgcaaggtctgggactggtcactatttgtgagcgtgaagatcctcgcgatgcctttgtgtccaataactatgacaatctggatgcgttaccggcaggcagtatcgtcgggacgtccagtttacgtcgccagtgccaactggctgaacgccgcccggatctgattatccgctccctgcgaggcaacgtcggcactcgcctgagtaaactggataacggcgaatacgatgccatcattcttgcggtagccggactaaaacgtttaggtctggagtcccgcattcgcgccgcattgccacccgagatttctcttccggcggtaggacaaggtgcggtgggtattgaatgccgccttgatgattctcgcactcgcgagctgcttgccgcgctgaatcaccacgaaactgcactgcgcgttaccgcagaacgcgccatgaatacccgtctcgaaggcggatgtcaggtgccaattggtagctacgccgagcttattgatggcgaaatctggctgcgtgcgttggtcggcgcgccggacggttcgcagattattcgcggtgaacgccgcggtgcgccgcaagatgccgaacaaatggggatttcgctggcagaagagctactgaataacggcgcgcgcgagatcctcgctgaagtctataacggagacgctccggcatgaagatctataaaaggaggaaaatatatgagtatcctggtcacccgcccgtctcccgctggagaagagttagtgagccgtctgcgcacactggggcaggtggcctggcattttccactgattgagttttctccgggtcgacaattaccacaacttgctgatcaactggcggcgctgggggagagcgatctgttgtttgccctctcgcaacacgcggttgcttttgcccaatcacagctgcatcagcaagatcgtaaatggccccgactacctgattatttcgccattggacgcaccaccgcactggcactacataccgtaagcggacagaagattctctacccgcaggatcgggaaatcagcgaagtcttgctacaattacctgaattacaaaatattgcgggcaaacgtgcgctgatattacgtggcaatggcggtcgtgagctaattggggataccctgacggcgcgcggtgctgaggtcactttttgtgaatgttatcaacgatgcgcaatccattacgatggtgcagaagaagcgatgcgctggcaatcccgcgaggtgacgacggtcgttgttaccagcggtgaaatgttgcagcaactctggtcgctgatcccacaatggtatcgtgagcactggttactacactgtcgactattggtcgtcagtgagcgtttggcgaaactcgcccgggaactgggctggcaagacattaaggtcgccgataacgctgacaacgatgcgcttttacgggcattacaataactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgacaagctgacgaccgggtctccgcaagtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcggtcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggcatgcagcgctcttccgcttcctcgctcactgactcgctacgctcggtcgttcgactgcggcgagcggtgtcagctcactcaaaagcggtaatacggttatccacagaatcaggggataaagccggaaagaacatgtgagcaaaaagcaaagcaccggaagaagccaacgccgcaggcgtttttccataggctccgcccccctgacgagcatcacaaaaatcgacgctcaagccagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgttggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccattggtaactgatttagaggactttgtcttgaagttatgcacctgttaaggctaaactgaaagaacagattttggtgagtgcggtcctccaacccacttaccttggttcaaagagttggtagctcagcgaaccttgagaaaaccaccgttggtagcggtggtttttctttatttatgagatgatgaatcaatcggtctatcaagtcaacgaacagctattccgttactctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattaatacgactcactata

SEQ ID NO: 59 - DNA, 8813 bp, synthetic construct
  source 1..8813: mol_type = other DNA
tctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatataccatgggtaccgaacttaaaaacgatcgttatctgcgggcgctgctgcgccagcccgttgatgtcactccagtatggatgatgcgccaggcgggtcgctatctaccggaatataaagccacgcgcgcccaggcgggcgattttatgtcgctgtgcaaaaacgccgagctggcgtgcgaagtgactttgcaaccgctgcgtcgctacccgctggatgcggcgatcctcttttccgatatcctcaccgtgccggacgcgatggggttagggctctattttgaagccggagaaggtccgcgttttacctcgccagtcacctgcaaagccgacgtcgataaactgccaattccggacccggaagatgagctgggttacgtgatgaacgcggtgcgtaccattcgtcgcgaactgaaaggcgaagtgccgctgattggtttttccggcagcccgtggacgctggcgacctacatggtggaaggcggcagcagcaaagcgttcaccgtgatcaaaaaaatgatgtatgccgatccgcaggcgctgcacgctctactcgataaactggcgaaaagcgtcactttgtatctgaatgcgcagattaaagccggtgctcaggcagtgatgattttcgacacctggggcggtgtgcttaccgggcgcgattatcaacagttctcgctctattacatgcataaaattgttgatggtttactgcgtgaaaacgacggtcgccgcgtaccggtcacgctgtttaccaaaggcggcggacagtggctggaagcgatggcagaaaccggttgcgatgcgttgggcctcgactggacaacggatatcgccgatgcgcgccgccgtgtgggcaataaagtcgcgttgcagggtaatatggatccgtcgatgctgtacgctccgcctgcccgcattgaagaagaagtagcgactatacttgcaggtttcggtcacggcgaaggtcatgtctttaaccttggtcacggcattcatcaggatgtgccgccagaacatgctggcgtattcgtggaggcagtgcatcgactgtctgaacaatatcaccgctaagagctcataaaaggaggaaaatatatgaaacccgacgcacaccaggttaaacagtttctgctcaaccttcaggatacgatttgtcagcagctgaccgccgtcgatggcgcagaatttgtcgaagatagttggcagcgcgaagctggcggcggcgggcgtagtcgggtgttgcgtaatggtggtgttttcgaacaggcaggcgtcaacttttcgcatgtccacggtgaggcgatgcctgcttccgccaccgctcatcgcccggaacttgccgggcgcagtttcgaggcgatgggcgtttcactggtagtgcatccgcataacccgtatgttcccaccagccacgcgaatgtgcggttttttattgccgaaaaaccgggtgccgatcccgtctggtggtttggcggcggcttcgatttaacccctttctatggttttgaagaagacgccattcactggcaccgcaccgcccgtgacctgtgcctgccatttggtgaagacgtttatccccgttacaaaaagtggtgcgacgattacttctacctcaaacatcgcaacgaacagcgcggtattggcgggctgttctttgatgatctgaacacgccagatttcgaccactgttttgcctttatgcaggcggtaggcaaaggctacaccgacgcttatttaccaattgtagagcgacgtaaagcgatggcctacggcgagcgcgagcgcaattttcagctctaccgtcgcggtcgttatgtcgagttcaatctggtctgggatcgcggcacgctgtttggcctgcaaactggcgggcgcaccgagtctatcctgatgtcaatgccgccactggtacgctgggaatatgattatcagccaaaagatggcagcccagaagcggcgttaagtgagtttattaaggtcagggattgggtgtaactgcagataaaaggaggaaaatatgtgaaaacattaattcttttctcaacaagggacggacaaacgcgcgagattgcctcctacctggcttcggaactgaaagaactggggatccaggcggatgtcgccaatgtgcaccgcattgaagaaccacagtgggaaaactatgaccgtgtggtcattggtgcttctattcgctatggtcactaccattcagcgttccaggaatttgtcaaaaaacatgcgacgcggctgaattcgatgccgagcgccttttactccgtgaatctggtggcgcgcaaaccggagaagcgtactccacagaccaacagctacgcgcgcaagtttctgatgaactcgcaatggcgtcccgatcgctgcgcggtcattgccggggcgctgcgttacccacgttatcgctggtacgaccgttttatgatcaagctgattatgaagatgtcaggcggtgaaacggatacgcgcaaagaagttgtctataccgattgggagcaggtggcgaatttcgcccgagaaatcgcccatttaaccgacaaaccgacgctgaaataagcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgcgtcagactaaaaccggtatcctgctggcaaacctgggtacgcccgatgcccccacacctgaagcggtaaaacgctatctgaaacaatttttaagcgacagacgcgtggttgatacctcacggttgttatggtggccattgctgcgcggcgtgattttgccgctgcgctcgccgcgtgtggcgaagctgtatgcctctgtctggatggaaggtggctcgccgctgatggtttacagccgtcagcaacagcaggcgctggcacaacgtttaccggagacgcccgtagcgctgggaatgagctacggctcgccatcactggaaagcgccgtagatgaactcctggcagagcatgtagatcatattgtggtgctgccgctttatccgcaatactcctgttcaacggtcggtgcggtatgggatgaactggcacgcattctggcgcgcaaacgtagcattccggggatatcgtttattcgtgattacgctgataaccacgattacattaatgcactggcgaacagcgtacgcgcttcttttgccaaacatggcgaaccggatctgctgctgctctcttatcatggcattccccagcgttatgcagatgaaggcgatgattacccgcaacgttgccgcacaacgactcgcgaactggcttccgcactggggatggcaccggaaaaagtgatgatgacctttcagtcgcgctttggtcgggaaccctggctgatgccttataccgacgaaacgctgaaaatgctcggagaaaaaggcgtaggtcatatacaggtgatgtgcccgggctttgctgcggattgtctggagacgctggaagagattgccgagcaaaaccgtgaggtcttcctcggtgccggcgggaaaaaatatgaatatattccagcgcttaatgccacgccggaacatattgaaatgatggctaatcttgttgccgcgtatcgctaactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacgg

SEQ ID NO: 60 - DNA, 1963 bp, synthetic construct
  source 1..1963: mol_type = other DNA
atgagcgttaacctggcgagccagctgcgtgaaggcaccaaaaaatctcactctatggcggaaaacgttggtttcgttaaatgcttcctgaaaggtgttgttgagaaaaacagctaccgtaaactggttggtaacctgtacttcgtttacagcgcgatggaagaagaaatggcgaaattcaaagatcacccgatcctgtctcacatctacttcccggaactgaaccgtaaacagtctctggaacaggatctgcagttctactatggtagcaactggcgtcaggaagttaaaatctctgcggcgggtcaggcgtacgttgatcgcgttcgtcaggttgcggcgaccgcgccggaactgctggttgcgcacagctacacccgttacctgggcgatctgagcggcggccagatcctgaagaaaatcgcgcagaacgcgatgaacctgcacgatggtggcaccgcgttctacgaattcgcagatatcgatgatgaaaaagcgttcaaaaacacctaccgtcaggctatgaacgatctgccgatcgatcaggcgaccgcggaacgtatcgttgatgaagcgaacgatgcgttcgcgatgaacatgaaaatgttcaacgaactggaaggtaacctgatcaaagcgatcggcatcatggttttcaactctctgacccgtcgtcgtagccagggcagcaccgaagttggtctggcgacctctgaaggctaagtcgacaagcttggtggcggcggcggatcaggtggtggtggttcaggtggtggtggttcaggtggtggtggttgtggtagcctgcgtgaatgtgaactgtatgttcagaaacataatattcaggccctgctgaaagatagcattgttcagctgtgtaccgcacgtccggaacgtccgatggcatttctgcgcgaatattttgaacgtctggaaaaagaagaagccaaacgaacagaaagtaatcgtattgtacacggccgcataatcgaaattaatacgactcactataggggaattgtgagcggataacaattccccatcttagtatattagttaagtataagaaggagatataggtggtggtggttcaggtggtggtggttcaggtggtggtggttgtggtctggaacagtatgcaaatcagctggcagatcagattatcaaagaagcaaccgaaggttgcgtcgacaagcttggtggcggcggcggatcaatggcggttaccgatctgtctctgaccaacagcagcctgatgccgaccctgaacccgatgatccagcagctggcgctggcgatcgcggcgtcttggcagagcctgccgctgaaaccgtaccagctgccggaagatctgggttacgttgaaggccgtctggaaggtgaaaaactggttatcgaaaaccgttgctaccagaccccgcagttccgtaaaatgcacctggaactggcaaaagtgggcaaaggtctggatatcctgcactgcgttatgttcccggaaccgctgtacggcctgccgctgttcggttgcgatatcgttgcgggtccgggtggtgttagcgcggctatcgcggatctgtctccgacccagtctgatcgtcagctgccggcggcgtaccagaaatctctggcggaactgggtcagccggaattcgaacagcagcgtgaactgccgccgtggggtgaaatcttctctgaatactgcctgttcattcgtccgtctaacgttaccgaagaagaacgtttcgttcagcgtgttgttgatttcctgcagatccactgccaccagagcatcgtggcggaaccgctgagcgaagcgcagaccctggaacaccgtcagggccagatccactactgccagcagcagcagaaaaacgataaaacccgtcgtgttctggaaaaagcgttcggcgaagcgtgggcggaacgttacatgtctcaggttctgttcgatgttatccagtaa